(19) United States
(12) Patent Application Publication
(10) Pub. No.: US 2026/0287571 A1
(43) Pub. Date: Sep. 24, 2026

(54) MACHINE LEARNING-BASED FUEL GAS COMPONENT ANALYSIS METHOD AND SYSTEM AND DEVICE

(71) Applicant: Harbin Institute of Technology, Harbin (CN)

(72) Inventors: Jianmin GAO, Harbin (CN); Qian Du, Harbin (CN); Heming Dong, Harbin (CN); Shijiu Ma, Harbin (CN); Ximei Li, Harbin (CN); Yu Zhang, Harbin (CN); Biao Huang, Harbin (CN)

(21) Appl. No.: 19/275,348

(22) Filed: Jul. 21, 2025

Related U.S. Application Data
(63) Continuation of application No. PCT/CN2025/092375, filed on Apr. 30, 2025.

(30) Foreign Application Priority Data
Feb. 11, 2025 (CN) 2025101483750

(51) Int. Cl. G01N 33/22; G06N 3/04

(52) U.S. Cl. CPC G01N 33/225; G06N 3/04

(57) ABSTRACT

The present application provides a machine learning-based fuel gas component analysis method, system and apparatus. The machine learning-based fuel gas component analysis method includes: obtaining the content of water produced by fuel gas combustion in a flue gas through the water content relationship according to the acquired flue gas carbon monoxide content, flue gas water content and air absolute humidity; obtaining the excess air coefficient through the excess air relationship according to the flue gas oxygen content, the flue gas carbon monoxide content and the flue gas carbon dioxide content; and inputting the excess air coefficient and the content of water produced by fuel gas combustion in the flue gas, as well as the acquired flue gas oxygen content, flue gas carbon dioxide content, flue gas carbon monoxide content, fuel gas density and combustion chamber temperature into a trained prediction model, and outputting the fuel gas component analysis result.

### CROSS-REFERENCE TO RELATED APPLICATIONS

This application is a bypass continuation application of PCT application no.: PCT/CN2025/092375. This application claims priorities from PCT Application PCT/CN2025/092375, filed Apr. 30, 2025, and from Chinese patent application 2025101483750, filed Feb. 11, 2025, the contents of which are incorporated herein in the entirety by reference.

### TECHNICAL FIELD

The present application relates to the technical field of fuel gas analysis, in particular to a machine learning-based fuel gas component analysis method, system and apparatus.

### BACKGROUND ART

In modern society, fuel gas, as an important form of energy, is widely applied in various fields such as household, industry, and commerce, and it is particularly important to analyze its components in order to ensure the safety, economy and environmental friendliness of the fuel gas.

In related technologies, instruments such as the gas chromatography device and the infrared spectroscopy device are commonly used for fuel gas component analysis, but fuel gas samples are often subjected to complex processing during analysis, which requires a lot of time and manpower, affecting the efficiency of fuel gas analysis.

### SUMMARY

The problem to be solved by the present application is how to improve the efficiency of fuel gas analysis.

To solve the above problem, the present application provides a machine learning-based fuel gas component analysis method, system and apparatus.

In a first aspect, the present application provides a machine learning-based fuel gas component analysis method, which includes:
acquiring the flue gas oxygen content, the flue gas carbon dioxide content, the flue gas carbon monoxide content and the flue gas water content in a flue gas, as well as the fuel gas density, the combustion chamber temperature and the air absolute humidity;
obtaining the content of water produced by fuel gas combustion in the flue gas through the water content relationship according to the flue gas carbon monoxide content, the flue gas water content and the air absolute humidity;
obtaining the excess air coefficient through the excess air relationship according to the flue gas oxygen content, the flue gas carbon monoxide content and the flue gas carbon dioxide content; and
inputting the excess air coefficient, the flue gas oxygen content, the flue gas carbon dioxide content, the flue gas carbon monoxide content, the content of water produced by fuel gas combustion in the flue gas, the fuel gas density and the combustion chamber temperature into a trained fuel gas component prediction model, and outputting a fuel gas component analysis result.

Optionally, the water content relationship satisfies:

$${C}_{{H}_{2} O}^{′} = {C}_{{H}_{2} 0} - {E}_{{H}_{2} O} \frac{2 \; 00 - {C}_{CO} - {C}_{{H}_{2} 0}}{200 - {E}_{{H}_{2} O}} ;$$

wherein,

$${C}_{{H}_{2} O}^{′}$$

is the content of water produced by fuel gas combustion in the flue gas; C_{H}_{₂}_{O} is the flue gas water content; E_{H}_{₂}_{O} is the air absolute humidity; and C_{CO} is the flue gas carbon monoxide content.

Optionally, the excess air relationship satisfies:

$$α = \frac{β}{β - ( 1 - β ) \frac{{C}_{{O}_{2}} - 0.5 {C}_{CO}}{100 - ( {C}_{{CO}_{2}} + {C}_{{O}_{2}} + {C}_{CO} )}} ;$$

wherein, α is the excess air coefficient; C_{O}_{₂} is the flue gas oxygen content; C_{CO} is the flue gas carbon monoxide content; C_{CO}_{₂} is the flue gas carbon dioxide content; and β is an oxygen content coefficient.

Optionally, a training process of the fuel gas component prediction model includes:
acquiring fuel gas component training data and an initial neural network, the fuel gas component training data comprising input data and label data, and the initial neural network comprising an input layer, a hidden layer, an output layer and a constraint layer;
transmitting the input data to the hidden layer through the input layer to obtain a hidden layer output result;
transmitting the hidden layer output result to the output layer to obtain an initial result outputted by the output layer;
transmitting the initial result to the constraint layer to obtain a prediction result outputted by the constraint layer; and
updating a hidden layer weight, a hidden layer bias, an output layer weight and an output layer bias in the initial neural network according to the prediction result and a comparison result of the corresponding label data to obtain the trained fuel gas component prediction model.

Optionally, the hidden layer output result satisfies:

$${H}_{j} = g ( {\sum }_{i = 1}^{n} {w}_{i j} {x}_{i} + {θ}_{j} ) ;$$

wherein, Hⱼ is the hidden layer output result of a j^{th} node in the hidden layer; wᵢⱼ is the hidden layer weight of the j^{th} node in the hidden layer corresponding to an i^{th} node in the input layer; xᵢ is an input value of the i^{th} node in the input layer; θⱼ is a bias corresponding to the j^{th} node in the hidden layer; g( ) is an activation function; and n is the quantity of nodes in the input layer.

Optionally, the output layer output result satisfies:

$${O}_{k} = {\sum }_{j = 1}^{l} {u}_{j k} {H}_{j} + {θ}_{k} ;$$

wherein, Oₖ is the output layer output result of a k^{th} node in the output layer; uⱼₖ is the output layer weight of the k^{th} node in the output layer corresponding to the j^{th} node in the hidden layer; Hⱼ is the hidden layer output result of the j^{th} node in the hidden layer; θₖ is a bias corresponding to the k^{th} node in the output layer; and 1 is the quantity of nodes in the hidden layer.

Optionally, the updated hidden layer weight satisfies:

$${w}_{i j}^{′} = {w}_{i j} + η {H}_{j} ( 1 - {H}_{j} ) {x}_{i} {\sum }_{k = 1}^{m} {u}_{j k} ( {Y}_{k} - {O}_{k} ) ;$$

the updated hidden layer bias satisfies:

$${θ}_{j}^{′} = {θ}_{j} + η {H}_{j} ( 1 - {H}_{j} ) {\sum }_{k = 1}^{m} {u}_{j k} ( {Y}_{k} - {O}_{k} ) ;$$

the updated output layer weight satisfies:

$${u}_{j k}^{′} = {u}_{j k} + η {H}_{j} ( {Y}_{k} - {O}_{k} ) ;$$

the updated output layer bias satisfies:

$${θ}_{k}^{′} = {θ}_{k} + η ( {Y}_{k} - {O}_{k} ) ;$$

wherein,

$${w}_{ij}^{′}$$

is the updated hidden layer weight of the j^{th} node in the hidden layer corresponding to the i^{th} node in the input layer; wᵢⱼ is the hidden layer weight of the j^{th} node in the hidden layer corresponding to the i^{th} node in the input layer; xᵢ is an input value of the i^{th} input node in the input layer; n is a preset parameter learning coefficient; Hⱼ is the hidden layer output result of the j^{th} node in the hidden layer;

$${u}_{jk}^{′}$$

is the updated output layer weight of the k^{th} node in the output layer corresponding to the j^{th} node in the hidden layer; uⱼₖ is the output layer weight of the k^{th} node in the output layer corresponding to the j^{th} node in the hidden layer; θₖ is the output layer output result of the k^{th} node in the output layer; Yₖ is the label data corresponding to the k^{th} node in the output layer; m is the quantity of nodes in the output layer;

$${θ}_{j}^{′}$$

is the updated hidden layer bias corresponding to the j^{th} node in the hidden layer; θⱼ is the hidden layer bias corresponding to the j^{th} node in the hidden layer;

$${θ}_{k}^{′}$$

is the updated output layer bias corresponding to the k^{th} node in the output layer; and θₖ is the output layer bias corresponding to the k^{th} node in the output layer.

Optionally, the constraint layer includes a component total content constraint, a carbon conservation constraint, a carbon-hydrogen ratio constraint, a fuel gas calorific value constraint, a fuel gas density constraint, a fuel gas component upper and lower limit constraint and an alkane component interval constraint; the initial result includes a fuel gas methane content, a fuel gas ethane content, a fuel gas propane content, and a fuel gas butane content; and the transmitting the initial result to the constraint layer to obtain the prediction result outputted by the constraint layer includes:
determining the initial result as the prediction result when the initial result satisfies the component total content constraint, the carbon conservation constraint, the carbon-hydrogen ratio constraint, the fuel gas calorific value constraint, the fuel gas density constraint, the fuel gas component upper and lower limit constraint and the alkane component interval constraint at the same time;
otherwise, determining a fuel gas component solution space according to the component total content constraint, the carbon conservation constraint, the carbon-hydrogen ratio constraint, the fuel gas calorific value constraint, the fuel gas density constraint, the fuel gas component upper and lower limit constraint and the alkane component interval constraint; and
performing optimization in the fuel gas component solution space according to the initial result, and determining a solution in the fuel gas component solution space that is closest to the initial result as the prediction result;
the component total content constraint satisfies:

$${C}_{{CH}_{4}} + {C}_{{C}_{2} {H}_{6}} + {C}_{{C}_{3} {H}_{8}} + {C}_{{C}_{4} {H}_{10}} < 100 ;$$

the carbon conservation constraint satisfies:

$${C}_{{CH}_{4}} + 2 {C}_{{C}_{2} {H}_{6}} + 3 {C}_{{C}_{3} {H}_{8}} + 4 {C}_{{C}_{4} {H}_{10}} < ( {C}_{{CO}_{2}} + {C}_{CO} ) \times {V}_{fg} ;$$

the carbon-hydrogen ratio constraint satisfies:

$${A}_{1} \leq \frac{4 {C}_{{CH}_{4}} + 6 {C}_{{C}_{2} {H}_{6}} + 8 {C}_{{C}_{3} {H}_{8}} + 10 {C}_{{C}_{4} {H}_{10}}}{{C}_{{CH}_{4}} + 2 {C}_{{C}_{2} {H}_{6}} + 3 {C}_{{C}_{3} {H}_{8}} + 4 {C}_{{C}_{4} {H}_{10}}} \leq {A}_{2} ;$$

the fuel gas calorific value constraint satisfies:

$${B}_{1} \leq 35.9 {C}_{{CH}_{4}} + 64.4 {C}_{{C}_{2} {H}_{6}} + 93.24 {C}_{{C}_{3} {H}_{8}} + 123.6 {C}_{{C}_{4} {H}_{10}} \leq {B}_{2} ;$$

the fuel gas density constraint satisfies:

$${C}_{1} \leq 0.72 {C}_{{CH}_{4}} + 1.36 {C}_{{C}_{2} {H}_{6}} + 2.01 {C}_{{C}_{3} {H}_{8}} + 2.7 {C}_{{C}_{4} {H}_{10}} \leq {C}_{2} ;$$

the fuel gas component upper and lower limit constraint satisfies:

$${ρ}_{min} \leq {C}_{{CH}_{4}} \leq {ρ}_{max} ;$$

$${σ}_{min} \leq {C}_{{C}_{2} {H}_{6}} \leq {σ}_{max} ;$$

$${τ}_{min} \leq {C}_{{C}_{3} {H}_{8}} \leq {τ}_{max} ;$$

$${φ}_{min} \leq {C}_{{C}_{4} {H}_{10}} \leq {φ}_{max} ;$$

the alkane component interval constraint satisfies:

$${C}_{{C}_{4} {H}_{10}} \leq {C}_{{C}_{2} {H}_{6}} ;$$

$${C}_{{C}_{3} {H}_{8}} \leq {C}_{{C}_{2} {H}_{6}} ;$$

the prediction result satisfies:

$$min f ( {C}_{i} ) = {\sum }_{i = 1}^{4} {( {C}_{i} - {C}_{i}^{′} )}^{2} , {C}_{i} ∈ {R}^{4} ;$$

wherein, C_{CH}_{₄} is the fuel gas methane content; C_{C}_{₂}_{H}_{₆} is the fuel gas ethane content; C_{C}_{₃}_{H}_{₈} is the fuel gas propane content; C_{C}_{₄}_{H}_{₁₀} is the fuel gas butane content; V_{fg} is the acquired volume of a flue gas generated by fuel gas combustion; A₁ is a preset carbon-hydrogen ratio lower limit; A₂ is a preset carbon-hydrogen ratio upper limit; B₁ is a preset fuel gas calorific value lower limit; B₂ is a preset fuel gas calorific value upper limit; C₁ is a preset fuel gas density lower limit; C₂ is a preset fuel gas density upper limit; min ƒ(Cᵢ) is a minimum value of an objective function ƒ(Cᵢ); Cᵢ is a content of an i^{th} component in a fuel gas component prediction result;

$${C}_{i}^{′}$$

is a content of an i^{th} component in a fuel gas component initial result; R⁴ is a four-dimensional fuel gas component solution space; ρₘᵢₙ is a lower limit of the fuel gas methane content; ρₘₐₓ is an upper limit of the fuel gas methane content; σₘᵢₙ is a lower limit of the fuel gas ethane content; σₘₐₓ is an upper limit of the fuel gas ethane content; τₘᵢₙ is a lower limit of the fuel gas propane content; τₘₐₓ is an upper limit of the fuel gas propane content; φₘᵢₙ is a lower limit of the fuel gas butane content; and φₘₐₓ is an upper limit of the fuel gas butane content.

In a second aspect, the present application provides a machine learning-based fuel gas component analysis system, which includes:
an acquisition module, configured to acquire the flue gas oxygen content, the flue gas carbon dioxide content, the flue gas carbon monoxide content and the flue gas water content in a flue gas, as well as the fuel gas density, the combustion chamber temperature and the air absolute humidity;
a first processing module, configured to obtain the content of water produced by fuel gas combustion in the flue gas through the water content relationship according to the flue gas carbon monoxide content, the flue gas water content and the air absolute humidity;
a second processing module, configured to obtain the excess air coefficient through the excess air relationship according to the flue gas oxygen content, the flue gas carbon monoxide content and the flue gas carbon dioxide content; and
a prediction module, configured to input the excess air coefficient, the flue gas oxygen content, the flue gas carbon dioxide content, the flue gas carbon monoxide content, the content of water produced by fuel gas combustion in the flue gas, the fuel gas density and the combustion chamber temperature into a trained fuel gas component prediction model, and output a fuel gas component analysis result.

In a third aspect, the present application provides an electronic device, including a memory and a processor, wherein
the memory is configured to have stored a computer program therein; and
the processor is configured to implement the machine learning-based fuel gas component analysis method in the first aspect while executing the computer program.

In a fourth aspect, the present application provides a computer-reasonable storage medium, configured to have stored a computer program therein, which, when being executed by a processor, implements the machine learning-based fuel gas component analysis method in the first aspect.

The machine learning-based fuel gas component analysis method and system and the device of the present application have the following beneficial effects: acquiring a plurality of key parameters such as the flue gas oxygen content, the flue gas carbon dioxide content, the flue gas carbon monoxide content and the flue gas water content in the flue gas, as well as the fuel gas density, the combustion chamber temperature and the air absolute humidity, such that repeated operations in the data acquisition process are reduced and the time is saved, thereby improving the overall analysis efficiency. The content of water produced by fuel gas combustion in the current flue gas is calculated through the preset water content relationship according to the flue gas carbon monoxide content, the flue gas water content and the air absolute humidity in the flue gas; and the excess air coefficient is quickly calculated through the excess air relationship according to the acquired flue gas oxygen content, flue gas carbon monoxide content and flue gas carbon dioxide content, which can quickly provide important parameters for the subsequent fuel gas component prediction without complex experimental devices or long-term reaction process, thereby accelerating the advancement of the analysis process. A plurality of fuel gas and flue gas parameters, including the excess air coefficient, are inputted into the trained fuel gas component prediction model, and the fuel gas component analysis result is quickly outputted. This model which is trained on a large amount of data has efficient computing capability and accurate prediction performance, and can analyze and process complex input parameters within a short time. Compared with the traditional analysis method, the present method introduces the neural network model, which greatly shortens the time required to predict the fuel gas components, significantly improves the analysis efficiency, also avoids the interference caused by different fuel gas components to the detection result during the fuel gas component detection, improves the accuracy of fuel gas component analysis, and ensures the physical authenticity of the fuel gas component analysis result by introducing various physical constraints.

### BRIEF DESCRIPTION OF THE DRAWINGS

FIG. 1 is a schematic flowchart of the machine learning-based fuel gas component analysis method provided by an embodiment of the present application;

FIG. 2 is a schematic structural diagram of the machine learning-based fuel gas component analysis system provided by an embodiment of the present application; and

FIG. 3 is a schematic structural diagram of an electronic device provided by an embodiment of the present application.

### DETAILED DESCRIPTION OF THE INVENTION

In order to make the above objects, features and advantages of the present application clearer, the specific embodiments of the present application are described in detail in conjunction with the accompanying drawings. Although some embodiments of the present application are shown in the drawings, it should be understood that the present application may be implemented in various forms and should not be limited by the embodiments described herein. Rather, these embodiments are provided for a more thorough and complete understanding of the present application. It should be understood that the accompanying drawings and embodiments of the present application are only used for an exemplary purpose, but not to limit the protection scope of the present application.

It should be understood that the steps described in the method embodiment of the present application may be performed in different sequences, and/or in parallel. In addition, the method embodiment may include additional steps and/or omit the steps shown in the execution. The scope of the present application is not limited in this aspect.

The term “comprising/including” and its variations as used herein are open inclusions, i.e., “including but not limited to”; the term “based on” is “at least partially based on”; the term “one embodiment” means “at least one embodiment”; the term “another embodiment” means “at least one additional embodiment”; the term “some embodiments” means “at least some embodiments”; and the term “optionally” means “optional embodiment”. The relevant definitions of other terms are given in the following descriptions. It should be noted that the concepts of “first”, “second” and the like mentioned in the present application are only used to distinguish different apparatuses, modules or units, but are not used to limit the order or interdependence of the functions performed by these apparatuses, modules or units.

It should be noted that the modifications of “one” and “a plurality of” mentioned in the present application are schematic but not restrictive, and those skilled in the art should understand that they should be understood as “one or more” unless otherwise expressly indicated in the context.

The names of messages or information exchanged between a plurality of apparatuses in the embodiments of the present application are used only for illustrative purposes, but not intended to limit the scope of such messages or information.

In related technologies, the main method for real-time measurement of components of natural gas burnt by a fuel gas device at present is to directly detect the components of the natural gas at an inlet end of the device. The commonly used online full-component detection methods for multi-component gases mainly include gas chromatography method and Fourier transform infrared spectroscopy method. The gas chromatography method can obtain physical parameters such as the composition, density, calorific value, etc. of natural gas. However, gas chromatograph requires a carrier gas to run with long time consumption for separation and detection, so it is impossible to monitor the gas components in real time. The infrared spectroscopy technology is fast in analysis speed, but cannot detect homonuclear diatomic molecules such as nitrogen and hydrogen. Since the natural gas is a mixture of various alkanes and other gases, it is inevitable that there will be a certain proportion of nitrogen and hydrogen in the natural gas, which will bring the problem of cross-sensitivity of detection results and also results in measurement interference between fuel gas components. Therefore, conventional fuel gas detection devices consume a lot of time when performing fuel gas component analysis, so the accuracy is difficult to guarantee.

In view of the problems existing in related technologies, the present embodiment provides a machine learning-based fuel gas component analysis method, system and apparatus.

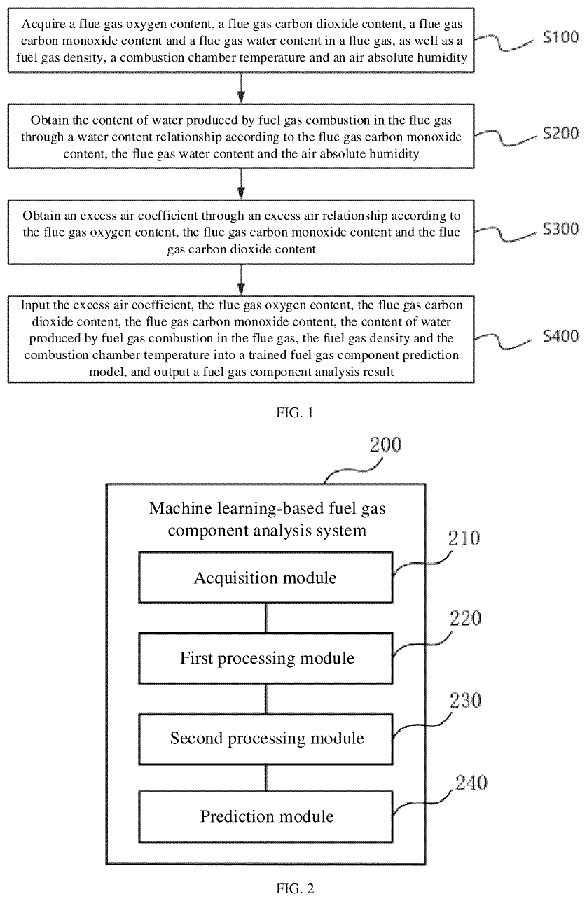

A shown in FIG. 1, an embodiment of the present application provides a machine learning-based fuel gas component analysis method, which includes:

S100, acquiring the flue gas oxygen content, the flue gas carbon dioxide content, the flue gas carbon monoxide content and the flue gas water content in a flue gas, as well as the fuel gas density, the combustion chamber temperature and the air absolute humidity.

It should be noted that, the present embodiment, by integrating energy science, chemical science, artificial intelligence and other research fields and taking the online identification of fuel gas calorific value changes as a starting point, provides a fuel gas component analysis method associated with fuel gas and flue gas dual systems based on carbon-hydrogen balance and chemical reaction heat correction, wherein the components of natural gas burned by a fuel gas device can be inversed by using operating parameters of the fuel gas device such as flue gas information, and the changes in the components of the natural gas used by the fuel gas device can be accurately analyzed.

Specifically, component data of the flue gas produced by the gas device by combustion of the natural gas, including the flue gas oxygen content, the flue gas carbon dioxide content, the flue gas carbon monoxide content and the flue gas water content, are acquired, while the fuel gas density of the fuel gas inputted to the fuel gas device and the combustion chamber temperature of a combustion chamber, as well as the air absolute humidity in the current air are acquired. Since a main component of the fuel gas is hydrocarbons in which carbon is converted into carbon dioxide and carbon monoxide when the fuel gas is in combustion. By measuring the content of carbon dioxide and carbon monoxide in a flue gas, the content of carbon in the fuel gas can be calculated, and then the proportion of a carbon-containing compound can be estimated. In the same way, since hydrogen produces water in combustion, the amount of water produced by fuel gas combustion in the flue gas can be calculated, and the content of hydrogen in the fuel gas can be estimated, helping determine the composition of various hydrocarbons in the fuel gas.

S200, obtaining the content of water produced by fuel gas combustion in the flue gas through the water content relationship according to the flue gas carbon monoxide content, the flue gas water content and the air absolute humidity.

S300, obtaining the excess air coefficient through the excess air relationship according to the flue gas oxygen content, the flue gas carbon monoxide content and the flue gas carbon dioxide content.

Specifically, the content of moisture produced by fuel gas combustion in the current flue gas, i.e., the content of water produced by fuel gas combustion in the flue gas, is calculated through the preset water content relationship according to the flue gas carbon monoxide content, the flue gas water content and the air absolute humidity in the flue gas; and the corresponding excess air coefficient is calculated through a preset excess air relationship according to the flue gas oxygen content, the flue gas carbon monoxide content and the flue gas carbon dioxide content in the flue gas; and the excess air content can reflect the degree of air supply and combustion completeness in combustion.

S400, inputting the excess air coefficient, the flue gas oxygen content, the flue gas carbon dioxide content, the flue gas carbon monoxide content, the content of water produced by fuel gas combustion in the flue gas, the fuel gas density and the combustion chamber temperature into a trained fuel gas component prediction model, and outputting a fuel gas component analysis result.

Specifically, the excess air coefficient, the content of various components in the flue gas (the flue gas oxygen content, the flue gas carbon dioxide content, the flue gas carbon monoxide content and the content of water produced by fuel gas combustion in the flue gas), the fuel gas density and the combustion chamber temperature are inputted into the trained fuel gas component prediction model to obtain an output result that satisfies constraints.

In the present embodiment, a plurality of key parameters such as the flue gas oxygen content, the flue gas carbon dioxide content, the flue gas carbon monoxide content and the flue gas water content in the flue gas, as well as the fuel gas density, the combustion chamber temperature and the air absolute humidity are acquired, such that repeated operations in the data acquisition process are reduced, and the time is saved, thereby improving the overall analysis efficiency. The content of water produced by fuel gas combustion in the current flue gas is calculated through the preset water content relationship according to the flue gas carbon monoxide content, the flue gas water content and the air absolute humidity in the flue gas; and the excess air coefficient is quickly calculated through the excess air relationship according to the acquired flue gas oxygen content, flue gas carbon monoxide content and flue gas carbon dioxide content, which can quickly provide important parameters for the subsequent fuel gas component prediction without complex experimental devices or long-term reaction process, thereby accelerating the advancement of the analysis process. A plurality of fuel gas and flue gas parameters, including the excess air coefficient, are inputted into the trained fuel gas component prediction model, and the fuel gas component analysis result is quickly outputted. This model which is trained on a large amount of data has efficient computing capability and accurate prediction performance, and can analyze and process complex input parameters within a short time. Compared with the traditional analysis method, the present method introduces the neural network model, which greatly shortens the time required to predict the fuel gas components, significantly improves the analysis efficiency, also avoids the interference caused by different fuel gas components to the detection result during the fuel gas component detection, improves the accuracy of fuel gas component analysis, and ensures the physical authenticity of the fuel gas component analysis result by introducing various physical constraints.

Optionally, the water content relationship satisfies:

$${C}_{{H}_{2} O}^{′} = {C}_{{H}_{2} 0} - {E}_{{H}_{2} O} \frac{200 - {C}_{CO} - {C}_{{H}_{2} 0}}{200 - {E}_{{H}_{2} O}} ;$$

wherein,

$${C}_{{H}_{2} O}^{′}$$

is the content of water produced by fuel gas combustion in the flue gas; C_{H}_{₂}₀ is the flue gas water content; E_{H}_{₂}_{O} is the air absolute humidity; and C_{CO} is the flue gas carbon monoxide content.

Optionally, the excess air relationship satisfies:

$$α = \frac{β}{β - ( 1 - β ) \frac{{C}_{{O}_{2}} - 0.5 {C}_{CO}}{100 - ( {C}_{{CO}_{2}} + {C}_{{O}_{2}} + {C}_{CO} )}} ;$$

wherein, α is the excess air coefficient; C_{O}_{₂} is the flue gas oxygen content; C_{CO} is the flue gas carbon monoxide content; C_{CO}_{₂} is the flue gas carbon dioxide content; and β is an oxygen content coefficient.

Optionally, a training process of the fuel gas component prediction model includes:
acquiring fuel gas component training data and an initial neural network, the fuel gas component training data including input data and label data, and the initial neural network including an input layer, a hidden layer, an output layer and a constraint layer;
transmitting the input data to the hidden layer through the input layer to obtain a hidden layer output result;
transmitting the hidden layer output result to the output layer to obtain an initial result outputted by the output layer;
transmitting the initial result to the constraint layer to obtain a prediction result outputted by the constraint layer; and
updating a hidden layer weight, a hidden layer bias, an output layer weight and an output layer bias in the initial neural network according to the prediction result and a comparison result of the corresponding label data to obtain the trained fuel gas component prediction model.

Specifically, firstly, fuel gas component input data is constructed according to historical data of the fuel gas device. These data are various characteristic information related to the fuel gas components, such as the flue gas oxygen content, the flue gas carbon dioxide content, the flue gas carbon monoxide content, the flue gas water content, the fuel gas density, the combustion chamber temperature and the air absolute humidity, etc., and these parameters reflect the fuel gas combustion process and related environmental information from different angles and provide an important basis for predicting the fuel gas components. A large number of representative training data samples can be collected to provide rich materials for a neural network to learn the relationships between the fuel gas components and these parameters. Corresponding to each set of training data, actual fuel gas component information (i.e., an expected output of model training), such as the specific content of various fuel gas components, which may include the fuel gas methane content, the fuel gas ethane content, the fuel gas propane content and the fuel gas butane content, is clearly given through the corresponding label data. These data are the goal of neural network learning. The neural network attempts to make the prediction result as close to these actual label data as possible by constantly adjusting their own parameters. The initial neural network includes an input layer, a hidden layer, an output layer, and a constraint layer. The input layer serves as an entrance of the neural network, and functions to receive training data. That is, the training data is inputted into the input layer as input data, and the training data is transmitted to the hidden layer. The quantity of neurons in the input layer usually corresponds to the quantity of features of the training data, ensuring that each input feature can be processed by the neural network. The hidden layer, as one of the core parts of the neural network, does not directly interact with external data, but performs complex nonlinear transformations on the training data internally. The hidden layer performs feature extraction and combination on the training data through a series of neurons and weights, and mines potential patterns and relationships in the data. The quantity of neurons in the hidden layer and the quantity of the hidden layers can be set according to actual needs. Different settings will affect the learning capability and expression capability of the neural network. The output layer receives the output result of the hidden layer and converts it into the initial result, that is, the initially predicted gas components. The quantity of neurons in the output layer corresponds to the types of gas components that needs to be predicted. An output value of each neuron represents a predicted content of a gas component. The constraint layer receives an initial result outputted by the output layer and determines whether the initial result satisfies constraints, so as to output a final prediction result through the constraint layer.

Exemplarily, the training data is transmitted to the hidden layer through the input layer to obtain a hidden layer output result. The data transmission process is as follows: the training data is transmitted into the input layer, and each input feature is then assigned to the corresponding neuron. The neuron multiplies the received data values by corresponding weights (an initial hidden layer weight may be randomly generated), and then the weighted results are summed. Next, this sum value is processed through an activation function. The activation function gives the neural network nonlinear properties, allowing the neural network to learn complex nonlinear relationships. Common activation functions include a Sigmoid function, a ReLU function, and the like. After being processed with the activation function, the obtained values are an output result of neurons in the hidden layer, all the neurons in the hidden layer are calculated according to this process, and a hidden layer output result is finally generated. This result contains training data information after preliminary processing and feature extraction.

Then, the hidden layer output result is transmitted to the output layer to obtain an output layer output result. The hidden layer output result, which serves as an input of the output layer, will also undergo a similar calculation process as the hidden layer. The neurons in the output layer multiply the received output values of the hidden layer by their respective weights (an initial output layer weight may be randomly generated), then summed and processed through an activation function (sometimes the output layer may use a special activation function; for example, a Softmax function is used for a multi-classification problem, and in the prediction of fuel gas components, if the fuel gas components are regarded as different categories, a similar function may also be used) to obtain an initial result outputted by the output layer. The initial result is transmitted to the constraint layer to determine whether the initial result satisfies a preset constraint condition in the constraint layer. If the initial result satisfies the preset constraint condition, the initial result is taken as a final prediction result; and if the initial result does not satisfy the preset constraint condition, optimization is performed in the fuel gas component solution space generated by the constraint conditions in a prediction layer, and a solution closest to the initial result is taken as a final prediction result. This prediction result is a prediction value of the neutral network for a fuel gas component corresponding to the current training data. Therefore, a hidden layer weight, a hidden layer bias, an output layer weight and an output layer bias in the initial neural network are updated according to the prediction result and a comparison result of the corresponding label data (i.e., a prediction result and an error of the corresponding expected output) to obtain the trained fuel gas component prediction model.

Further, in the event of performing the error calculation, the prediction result outputted by the constraint layer is compared with the label data (i.e., actual fuel gas component data). An error between the prediction result and the label data is calculated through a loss function (e.g., an mean square deviation error, which is used to measure the degree of difference between a predicted value and a true value). The error value reflects a deviation degree between the current neural network prediction result and the actual situation, and the smaller the error, the closer the prediction result is to the true value. A preset error threshold may be set. When the error is less than this error threshold, the prediction result outputted by the model is determined to satisfy a requirement, and an initial neural network model that satisfies the requirement is determined as the finally trained fuel gas component prediction model.

Weight and bias updates: in order to reduce the error, the hidden layer weight and the output layer weight, as well as the hidden layer bias and the output layer bias in the neural network, need to be adjusted. By inputting the training data into the neural network for a plurality of times, the error is calculated and the weights and biases of the hidden layer and the output layer are updated, such that the prediction result of the neural network will gradually approach the actual fuel gas components to finally obtain the trained fuel gas component prediction model. This model can make more accurate fuel gas component prediction according to the inputted training data.

In the present optional embodiment, by training the fuel gas component prediction model, the prediction model can process multi-dimensional complex fuel gas data in parallel, quickly mine a nonlinear relationship between parameters and fuel gas components, and perform quick prediction based on existing learning results by virtue of strong pattern recognition capability, thereby saving the analysis time. The weights are automatically updated during training to reduce manual interventions and quickly optimize the model, and can adapt to different fuel gas characteristics, without the need to develop specific processes for various types of fuel gases, thereby improving the overall efficiency.

Optionally, the hidden layer output result satisfies:

$${H}_{j} = g ( {\sum }_{i = 1}^{n} {w}_{ij} {x}_{i} + {θ}_{j} ) ;$$

wherein, Hⱼ is the hidden layer output result of a j^{th} node in the hidden layer; wᵢⱼ is the hidden layer weight of the j^{th} node in the hidden layer corresponding to an i^{th} node in the input layer; xᵢ is an input value of the i^{th} node in the input layer; θⱼ is a bias corresponding to the j^{th} node in the hidden layer; g( ) is an activation function; and n is the quantity of nodes in the input layer.

Optionally, the output layer output result satisfies:

$${O}_{k} = {\sum }_{j = 1}^{l} {u}_{jk} {H}_{j} + {θ}_{k} ;$$

wherein, θₖ is the output layer output result of a k^{th} node in the output layer; uⱼₖ is the output layer weight of the k^{th} node in the output layer corresponding to the j^{th} node in the hidden layer; Hⱼ is the hidden layer output result of the j^{th} node in the hidden layer; θₖ is a bias corresponding to the k^{th} node in the output layer; and 1 is the quantity of nodes in the hidden layer.

Optionally, the updated hidden layer weight satisfies:

$${w}_{ij}^{′} = {w}_{ij} + η {H}_{j} ( 1 - {H}_{j} ) {x}_{i} {\sum }_{k = 1}^{m} {u}_{jk} ( {Y}_{k} - {O}_{k} ) ;$$

the updated hidden layer bias satisfies:

$${θ}_{j}^{′} = {θ}_{j} + η {H}_{j} ( 1 - {H}_{j} ) {\sum }_{k = 1}^{m} {u}_{jk} ( {Y}_{k} - {O}_{k} ) ;$$

the updated output layer weight satisfies:

$${u}_{jk}^{′} = {u}_{jk} + η {H}_{j} ( {Y}_{k} - {O}_{k} ) ;$$

the updated output layer bias satisfies:

$${θ}_{k}^{′} = {θ}_{k} + η ( {Y}_{k} - {O}_{k} ) ;$$

wherein,

$${w}_{ij}^{′}$$

is the updated hidden layer weight of the j^{th} node in the hidden layer corresponding to the i^{th} node in the input layer; wᵢⱼ is the hidden layer weight of the j^{th} node in the hidden layer corresponding to the i^{th} node in the input layer; xᵢ is an input value of the i^{th} input node in the input layer; η is a preset parameter learning coefficient; Hⱼ is the hidden layer output result of the j^{th} node in the hidden layer,

$${u}_{jk}^{′}$$

is the updated output layer weight of the k^{th} node in the output layer corresponding to the j^{th} node in the hidden layer; uⱼₖ is the output layer weight of the k^{th} node in the output layer corresponding to the j^{th} node in the hidden layer; θₖ is the output layer output result of the k^{th} node in the output layer; Yₖ is the label data corresponding to the k^{th} node in the output layer; m is the quantity of nodes in the output layer;

$${θ}_{j}^{′}$$

is the hidden layer bias corresponding to the j^{th} node in the hidden layer; θⱼ is the hidden layer bias corresponding to the j^{th} node in the hidden layer;

$${θ}_{k}^{′}$$

is the updated output layer bias corresponding to the k^{th} node in the output layer; and θₖ is the output layer bias corresponding to the k^{th} node in the output layer.

Optionally, the constraint layer includes a component total content constraint, a carbon conservation constraint, a carbon-hydrogen ratio constraint, a fuel gas calorific value constraint, a fuel gas density constraint, a fuel gas component upper and lower limit constraint and an alkane component interval constraint; the initial result includes a fuel gas methane content, a fuel gas ethane content, a fuel gas propane content, and a fuel gas butane content; and the transmitting the initial result to the constraint layer to obtain the prediction result outputted by the constraint layer includes:
determining the initial result as the prediction result when the initial result satisfies the component total content constraint, the carbon conservation constraint, the carbon-hydrogen ratio constraint, the fuel gas calorific value constraint, the fuel gas density constraint, the fuel gas component upper and lower limit constraint and the alkane component interval constraint at the same time;
otherwise, determining a fuel gas component solution space according to the component total content constraint, the carbon conservation constraint, the carbon-hydrogen ratio constraint, the fuel gas calorific value constraint, the fuel gas density constraint, the fuel gas component upper and lower limit constraint and the alkane component interval constraint; and
performing optimization in the fuel gas component solution space according to the initial result, and determining a solution in the fuel gas component solution space that is closest to the initial result as the prediction result;
the component total content constraint satisfies:

$${C}_{{CH}_{4}} + {C}_{{C}_{2} {H}_{6}} + {C}_{{C}_{3} {H}_{8}} + {C}_{{C}_{4} {H}_{10}} < 100 ;$$

the carbon conservation constraint satisfies:

$${C}_{{CH}_{4}} + 2 {C}_{{C}_{2} {H}_{6}} + 3 {C}_{{C}_{3} {H}_{8}} + 4 {C}_{{C}_{4} {H}_{10}} < ( {C}_{{CO}_{2}} + {C}_{CO} ) \times {V}_{fg} ;$$

the carbon-hydrogen ratio constraint satisfies:

$${A}_{1} \leq \frac{4 {C}_{{CH}_{4}} + 6 {C}_{{C}_{2} {H}_{6}} + 8 {C}_{{C}_{3} {H}_{8}} + 10 {C}_{{C}_{4} {H}_{10}}}{{C}_{{CH}_{4}} + 2 {C}_{{C}_{2} {H}_{6}} + 3 {C}_{{C}_{3} {H}_{8}} + 4 {C}_{{C}_{4} {H}_{10}}} \leq {A}_{2} ;$$

the fuel gas calorific value constraint satisfies:

$${B}_{1} \leq 3 5.9 {C}_{{CH}_{4}} + 64.4 {C}_{{C}_{2} {H}_{6}} + 93.24 {C}_{{C}_{3} {H}_{8}} + 12 3.6 {C}_{{C}_{4} {H}_{10}} \leq {B}_{2} ;$$

the fuel gas density constraint satisfies:

$${C}_{1} \leq 0.72 {C}_{{CH}_{4}} + 1.36 {C}_{{C}_{2} {H}_{6}} + 2.01 {C}_{{C}_{3} {H}_{8}} + 2.7 {C}_{{C}_{4} {H}_{10}} \leq {C}_{2} ;$$

the fuel gas component upper and lower limit constraint satisfies:

$${ρ}_{min} \leq {C}_{{CH}_{4}} \leq {ρ}_{max} ;$$

$${σ}_{min} \leq {C}_{{C}_{2} {H}_{6}} \leq {σ}_{max} ;$$

$${τ}_{min} \leq {C}_{{C}_{3} {H}_{8}} \leq {τ}_{max} ;$$

$${φ}_{min} \leq {C}_{{C}_{4} {H}_{10}} \leq {φ}_{max} ;$$

the alkane component interval constraint satisfies:

$${C}_{{C}_{4} {H}_{10}} \leq {C}_{{C}_{2} {H}_{6}} ;$$

$${C}_{{C}_{3} {H}_{8}} \leq {C}_{{C}_{2} {H}_{6}} ;$$

the prediction result satisfies:

$$min f ( {C}_{i} ) = {\sum }_{i = 1}^{4} {( {C}_{i} - {C}_{i}^{′} )}^{2} , {C}_{i} ∈ {R}^{4} ;$$

wherein, C_{CH}_{₄} is the fuel gas methane content; C_{C}_{₂}_{H}_{₆} is the fuel gas ethane content; C_{C}_{₃}_{H}_{₈} is the fuel gas propane content; C_{C}_{₄}_{H}_{₁₀} is the fuel gas butane content; V_{fg} is the acquired volume of a flue gas generated by fuel gas combustion; A₁ is a preset carbon-hydrogen ratio lower limit; A₂ is a preset carbon-hydrogen ratio upper limit; B₁ is a preset fuel gas calorific value lower limit; B₂ is a preset fuel gas calorific value upper limit; C₁ is a preset fuel gas density lower limit; C₂ is a preset fuel gas density upper limit; min ƒ(Cᵢ) is a minimum value of an objective function ƒ(Cᵢ); Cᵢ is a content of an i^{th} component in a fuel gas component prediction result;

$${C}_{i}^{′}$$

is a content of an i^{th} component in a fuel gas component initial result; and R⁴ is a four-dimensional fuel gas component solution space.

In the present optional embodiment, according to the component total content constraint, the carbon conservation constraint, the carbon-hydrogen ratio constraint, the fuel gas calorific value constraint, the fuel gas density constraint, the fuel gas component upper and lower limit constraint and an alkane component interval constraint of the constraint layer, whether the initial result outputted by the output layer satisfies the above constraint conditions at the same time is determined; and if this initial result satisfies the above constraint conditions, the initial result is used as a prediction result outputted by the initial neural network. If this initial result does not satisfy the above constraint conditions, a corresponding fuel gas component solution space is determined according to the component gross constraint, the carbon conservation constraint, the carbon-hydrogen ratio constraint, the fuel gas calorific value constraint, the fuel gas density constraint, the fuel gas component upper and lower limit constraint and the alkane component interval constraint. This fuel gas component solution space may include a value range of each fuel gas component value, and then the optimization is performed in the fuel gas component solution space according to the initial result that does not satisfy the constraint conditions, that is, an optimal solution closest to this initial result is found, such that the optimal solution closest to the initial result, which is obtained by optimization, is taken as the finally outputted prediction result. By performing optimization in the fuel gas component solution space, the optimal solution, that is, the predicted result, which is closest to the initial result, can be quickly determined to complete fuel gas component analysis quickly, thereby improving the efficiency and accuracy of fuel gas component analysis.

It should be noted that the component total content constraint means that a sum of the contents of methane, ethane, propane and butane is less than 100%, following conservation of total mass since there are other substances (e.g., carbon dioxide and other alkanes) in the fuel gas in addition to methane, ethane, propane and butane; the carbon conservation constraint means that an amount of carbon before and after the fuel gas reaction is less than an amount of carbon outputted in an outlet flue gas, so as to ensure that the prediction result is reasonable and accurate at the level of chemical elements; the carbon-hydrogen ratio constraint means that different fuel gases have specific carbon-hydrogen ratio ranges, so as to determine whether predicted components conform to the characteristics of fuel gas types; the fuel gas calorific value constraint means that the heat released by unit fuel gas combustion should be within a specified range to meet the needs of different uses; the fuel gas density constraint means that the fuel gas density is related to components under a specific temperature and pressure, which affects practical applications and can verify the reasonability of the components; the initial fuel gas prediction result can be determined as a constraint fuel gas component prediction result only when the initial fuel gas prediction result satisfies all the above constraints at the same time, providing a reliable basis for all aspects of the fuel gas; and the fuel gas component upper and lower limit constraint means that the upper and lower limits of the fuel gas methane content, the fuel gas ethane content, the fuel gas propane content and the fuel gas butane content that satisfy requirements are measured through a large number of experiments.

Specifically, the fuel gas component upper and lower limit constraint may be:

$$79 \leq {C}_{{CH}_{4}} \leq 98.5 ;$$

$$0.05 \leq {C}_{{C}_{2} {H}_{6}} \leq 10 ;$$

$$0 \leq {C}_{{C}_{3} {H}_{8}} \leq 5 ;$$

$$0 \leq {C}_{{C}_{4} {H}_{10}} \leq 0.85 .$$

Exemplarily, a fuel gas component detection device may be composed of an online detection module, a data storage module, a fuel gas component inversion module, and a fuel gas information output module. The online detection module includes measurement apparatuses for flue gas components, a flue gas humidity, an air humidity, a fuel gas density and a combustion chamber temperature, which are configured to measure the contents of oxygen, carbon dioxide, carbon monoxide and moisture in the flue gas, as well as a content of moisture, a fuel gas flow, a fuel gas density, an air flow and other parameters in air, providing data support for subsequent analysis. Based on the conservation of mass and energy, the content of moisture produced by fuel gas combustion is measured through a difference between the contents of moisture in the flue gas and moisture in the air. A fuel gas carbon-hydrogen ratio may be calculated by combining the contents of carbon dioxide and carbon monoxide in the flue gas to establish a carbon-hydrogen balance relationship between the fuel gas and the flue gas. The correlations between the combustion chamber temperature and the air flow as well as the fuel gas calorific value are used to construct the relationships between fuel gas chemical reaction heat and the combustion chamber temperature, and these relationships provide arguments for machine learning and constrain the output results. Based on the carbon-hydrogen balance and the chemical reaction heat correction, as well as a database constructed from relevant data, a flue gas information inversion fuel gas component model is constructed by using a machine learning BP neural network algorithm and a convex optimization theory. The specific steps include: reading data, setting up training and test data, constructing a BP neural network (setting the quantity of nodes in each layer, an excitation function, weights, biases, a network performance function, a convex optimization problem constraint condition and an objective function, etc.), and training the neural network (calculating outputs and errors of the hidden layer and the output layer, and updating the weights and thresholds until the error is minimal). A calorific value of a mixed fuel gas is calculated according to fuel gas components and their respective calorific values estimated by the inversion module, and the estimation results of the fuel gas components and calorific values are outputted to the user.

Further, the prediction result may be determined based on a preset objective function ƒ(Cᵢ), where a Hessian matrix in the objective function is positively definite, so that the objective function is convex. All of the constraint conditions are linear, and are thus convex. Since both the objective function and the constraint conditions are convex, the optimization problem is a convex optimization problem. An important feature of the convex optimization problem is that a local optimal solution is also a global optimal solution, which makes a solution result more reliable. The objective function is a polynomial function. The polynomial function is differentiable everywhere. Therefore, the objective function is differentiable at the optimal solution. All of the constraint conditions are linear, and the linear functions are differentiable everywhere. Therefore, the constraint condition is differentiable at the optimal solution.

Therefore, both the objective function and the constraint conditions are differentiable at the optimal solution, i.e., the optimization problem is a differentiable convex optimization problem. This means that at the optimal solution, gradient information may be used to analyze and solve the problem, which is the basis for applying a Karush Kuhn Tucker (KKT) condition. At the same time, it also means that it is feasible to introduce the convex optimization process into the neural network process as a constraint layer, because the convex optimization process will not interrupt an error backpropagation process in the neural network training process. Since the constraint condition carried by the convex optimization problem in the present embodiment is an inequality constraint, the KKT condition is used to solve the optimal solution, while the KKT condition is a standard method for solving the optimization problem with the inequality constraint.

Exemplarily, a gradient condition means that a linear combination of a gradient of the objective function and a gradient of the constraint condition is zero.

$$∇ f ( {C}_{i} ) + {\sum }_{r = 1}^{T} {λ}_{r} ∇ {g}_{r} ( {C}_{i} ) = 0$$

$${C}_{i} ∈ {R}^{4} ;$$

wherein, ƒ(Cᵢ) is the objective function; gᵣ(Cᵢ) is a r^{th} constraint condition; Cᵢ is a content of the i^{th} component in the fuel gas component prediction result, and T is the quantity of the constraint conditions.

The original feasibility means that all constraint conditions must be satisfied.
gᵣ(Cᵢ)≤0, where i=1, 2, 3, 4 and r=1, 2, . . . , 18, i.e., 18 constraint conditions; and
the duality feasibility means that a Lagrangian multiplier λᵣ must be non-negative.
λᵣ≥0, where r=1, 2, . . . , 18; and
a complementary relaxation condition means that a product of the Lagrangian multiplier and the constraint value is zero.
λᵣ∇gᵣ(Cᵢ)=0, where i=1, 2, 3, 4 and j=1, 2, . . . , 18.

By analyzing the four parts of the KKT condition, the optimal solution can be solved. In addition, the objective function achieves a minimum value at the optimal solution while satisfying all constraint conditions.

As shown in FIG. 2, a machine learning-based fuel gas component analysis system 200 provided by an embodiment of the present application includes:

an acquisition module 210, configured to acquire a flue gas oxygen content, a flue gas carbon dioxide content, a flue gas carbon monoxide content and a flue gas water content in a flue gas, as well as a fuel gas density, a combustion chamber temperature and an air absolute humidity;

a first processing module 220, configured to obtain a content of water produced by fuel gas combustion in the flue gas through a water content relationship according to the flue gas carbon monoxide content, the flue gas water content and the air absolute humidity;

a second processing module 230, configured to obtain an excess air coefficient through an excess air relationship according to the flue gas oxygen content, the flue gas carbon monoxide content and the flue gas carbon dioxide content; and

a prediction module 240, configured to input the excess air coefficient, the flue gas oxygen content, the flue gas carbon dioxide content, the flue gas carbon monoxide content, the content of water produced by fuel gas combustion in the flue gas, the fuel gas density and the combustion chamber temperature into a trained fuel gas component prediction model, and output a fuel gas component analysis result.

The machine learning-based fuel gas component analysis system of the present embodiment is configured to implement the above-mentioned machine learning-based fuel gas component analysis method, and has the same advantages over the prior art as the advantages of the above-mentioned machine learning-based fuel gas component analysis method, which will not be repeated herein.

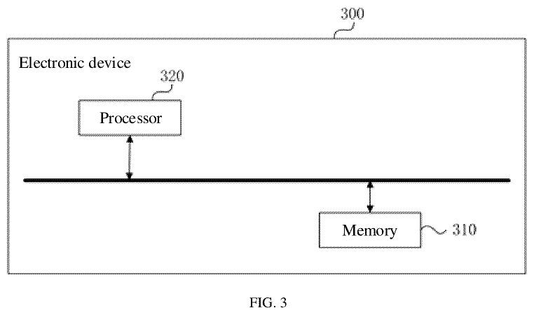

As shown in FIG. 3, an electronic device 300 provided by an embodiment of the present application includes a memory 310 and a processor 320, wherein the memory 310 is configured to have stored a computer program therein, and the processor 320 is configured to implement the above-mentioned machine learning-based fuel gas component analysis method while executing the computer program.

Alternatively, an electronic device 300 includes a memory 310 and a processor 320 coupled to the memory 310, wherein the memory 310 is configured to have stored a computer program therein, and the processor 320 is configured to implement the following operations while executing the computer program:
acquiring the flue gas oxygen content, the flue gas carbon dioxide content, the flue gas carbon monoxide content and the flue gas water content in a flue gas, as well as the fuel gas density, the combustion chamber temperature and the air absolute humidity;
obtaining the content of water produced by fuel gas combustion in the flue gas through the water content relationship according to the flue gas carbon monoxide content, the flue gas water content and the air absolute humidity;
obtaining the excess air coefficient through the excess air relationship according to the flue gas oxygen content, the flue gas carbon monoxide content and the flue gas carbon dioxide content; and
inputting the excess air coefficient, the flue gas oxygen content, the flue gas carbon dioxide content, the flue gas carbon monoxide content, the content of water produced by fuel gas combustion in the flue gas, the fuel gas density and the combustion chamber temperature into a trained fuel gas component prediction model, and outputting a fuel gas component analysis result.

An embodiment of the present application provides a computer-reasonable storage medium, configured to have stored a computer program therein, which, when being executed by a processor, implements the above-mentioned machine learning-based fuel gas component analysis method.

Alternatively, a non-transitory computer-readable storage medium is configured to have stored a computer program therein, which, when being executed by a processor, causes the processor to perform the following operations:
acquiring the flue gas oxygen content, the flue gas carbon dioxide content, the flue gas carbon monoxide content and the flue gas water content in a flue gas, as well as the fuel gas density, the combustion chamber temperature and the air absolute humidity;
obtaining the content of water produced by fuel gas combustion in the flue gas through the water content relationship according to the flue gas carbon monoxide content, the flue gas water content and the air absolute humidity;
obtaining the excess air coefficient through the excess air relationship according to the flue gas oxygen content, the flue gas carbon monoxide content and the flue gas carbon dioxide content; and
inputting the excess air coefficient, the flue gas oxygen content, the flue gas carbon dioxide content, the flue gas carbon monoxide content, the content of water produced by fuel gas combustion in the flue gas, the fuel gas density and the combustion chamber temperature into a trained fuel gas component prediction model, and outputting a fuel gas component analysis result.

The electronic device 300 that may be used as a server or client of the present application will now be described, which may be an example of a hardware device that can be applied to various aspects of the present application. The electronic device 300 is intended to represent various forms of digital-electronic computer devices, such as laptops, desktop computers, workbenches, personal digital assistants, servers, blade servers, mainframe computers, and other appropriate computers. The electronic device 300 may also represent various forms of mobile devices, such as personal digital assistants, cellular phones, smart phones, wearable devices and other similar computing apparatuses. The parts shown herein, their connections and relationships, and their functions are for example only and are not intended to limit the implementation of the present application described and/or required herein.

The electronic device 300 may include a computing unit, which may perform various appropriate actions and processing according to a computer program stored in a read-only memory (ROM) or a computer program that is loaded into a random access memory (RAM) from a storage unit. The RAM is further configured to have stored various programs and data required for the operations of a storage device. The computing unit, the ROM and the RAM are connected to each other via a bus. An input/output (I/O) interface is also connected to the bus.

A person with ordinary skill in the art may understand that all or some of procedures of the method in the foregoing embodiments may be implemented by a computer program instructing relevant hardware. The program may be stored in a computer-readable storage medium. When the program is executed, the procedures of the foregoing method embodiments may be implemented. The storage medium may be a magnetic disk, an optical disk, a read-only memory (ROM), a random access memory (RAM), or the like. In the present application, the units described as separate components may or may not be physically separated, and the components for unit description may or may not be physical units, that is, may be located in one place or distributed on a plurality of network units. Part or all of the units can be selected according to actual needs to achieve the objects of the embodiment solutions of the present application. In addition, all functional units in the embodiments of the present application can be integrated into one processing unit. Alternatively, each unit may exist physically independently. Alternatively, two or more units may be integrated into one unit. The above integrated units can be implemented in the form of hardware or software function units.

Although the present application discloses the following content, the protection scope of the present application is not limited to this. Those skilled in the art can make changes and modifications without departing from the spirit and scope of the present application, and these changes and modifications all fall within the protection scope of the present application.

1. A machine learning-based fuel gas component analysis method, comprising:
acquiring the flue gas oxygen content, the flue gas carbon dioxide content, the flue gas carbon monoxide content and the flue gas water content in a flue gas, as well as the fuel gas density, the combustion chamber temperature and the air absolute humidity;
obtaining the content of water produced by fuel gas combustion in the flue gas through the water content relationship according to the flue gas carbon monoxide content, the flue gas water content and the air absolute humidity;
obtaining the excess air coefficient through the excess air relationship according to the flue gas oxygen content, the flue gas carbon monoxide content and the flue gas carbon dioxide content; and
inputting the excess air coefficient, the flue gas oxygen content, the flue gas carbon dioxide content, the flue gas carbon monoxide content, the content of water produced by fuel gas combustion in the flue gas, the fuel gas density and the combustion chamber temperature into a trained fuel gas component prediction model, and outputting a fuel gas component analysis result.

2. The machine learning-based fuel gas component analysis method according to claim 1, wherein the water content relationship satisfies:
$${C}_{{H}_{2} O}^{′} = {C}_{{H}_{2} O} - {E}_{{H}_{2} O} \frac{200 - {C}_{CO} - {C}_{{H}_{2} O}}{200 - {E}_{{H}_{2} O}} ;$$
wherein, C_{H}_{₂}_{O} is the content of water produced by fuel gas combustion in the flue gas; C_{H}_{₂}₀ is the flue gas water content; E_{H}_{₂}_{O} is the air absolute humidity; and C_{CO} is the flue gas carbon monoxide content.

3. The machine learning-based fuel gas component analysis method according to claim 1, wherein the excess air relationship satisfies:
$$α = \frac{β}{β - ( 1 - β ) \frac{{C}_{{O}_{2}} - 0.5 {C}_{CO}}{100 - ( {{C}_{CO}}_{2} + {C}_{{O}_{2}} + {C}_{CO} )}} ;$$
wherein, α is the excess air coefficient; C_{O}_{₂} is the flue gas oxygen content; C_{CO} is the flue gas carbon monoxide content; C_{CO}_{₂} is the flue gas carbon dioxide content; and β is an oxygen content coefficient.

4. The machine learning-based fuel gas component analysis method according to claim 1, wherein a training process of the fuel gas component prediction model comprises:
acquiring fuel gas component training data and an initial neural network, the fuel gas component training data comprising input data and label data, and the initial neural network comprising an input layer, a hidden layer, an output layer and a constraint layer;
transmitting the input data to the hidden layer through the input layer to obtain a hidden layer output result;
transmitting the hidden layer output result to the output layer to obtain an initial result outputted by the output layer;
transmitting the initial result to the constraint layer to obtain a prediction result outputted by the constraint layer; and
updating a hidden layer weight, a hidden layer bias, an output layer weight and an output layer bias in the initial neural network according to the prediction result and a comparison result of the corresponding label data to obtain the trained fuel gas component prediction model.

5. The machine learning-based fuel gas component analysis method according to claim 4, wherein the hidden layer output result satisfies:
$${H}_{j} = g ( {\sum }_{i = 1}^{n} {w}_{ij} {x}_{i} + {θ}_{j} ) ;$$
wherein, Hⱼ is the hidden layer output result of a j^{th} node in the hidden layer; wᵢⱼ is the hidden layer weight of the j^{th} node in the hidden layer corresponding to an i^{th} node in the input layer; xᵢ is an input value of the i^{th} node in the input layer; θⱼ is a bias corresponding to the j^{th} node in the hidden layer; g( ) is an activation function; and n is the quantity of nodes in the input layer.

6. The machine learning-based fuel gas component analysis method according to claim 4, wherein the output layer output result satisfies:
$${O}_{k} = {\sum }_{j = 1}^{l} {u}_{jk} {H}_{j} + {θ}_{k} ;$$
wherein, Oₖ is the output layer output result of a k^{th} node in the output layer; uⱼₖ is the output layer weight of the k^{th} node in the output layer corresponding to the j^{th} node in the hidden layer; Hⱼ is the hidden layer output result of the j^{th} node in the hidden layer; θₖ is a bias corresponding to the k^{th} node in the output layer; and 1 is the quantity of nodes in the hidden layer.

7. The machine learning-based fuel gas component analysis method according to claim 5, wherein the updated hidden layer weight satisfies:
$${w}_{ij}^{′} = {w}_{ij} + η {H}_{j} ( 1 - {H}_{j} ) {x}_{i} {\sum }_{k = 1}^{m} {u}_{jk} ( {Y}_{k} - {O}_{k} ) ;$$
the updated hidden layer bias satisfies:
$${θ}_{j}^{′} = {θ}_{j} + η {H}_{j} ( 1 - {H}_{j} ) {\sum }_{k = 1}^{m} {u}_{jk} ( {Y}_{k} - {O}_{k} ) ;$$
the updated output layer weight satisfies:
$${u}_{jk}^{′} = {u}_{jk} + η {H}_{j} ( {Y}_{k} - {O}_{k} ) ;$$
the updated output layer bias satisfies:
$${θ}_{k}^{′} = {θ}_{k} + η ( {Y}_{k} - {O}_{k} ) ;$$
wherein,
$${w}_{ij}^{′}$$
is the updated hidden layer weight of the j^{th} node in the hidden layer corresponding to the i^{th} node in the input layer; wᵢⱼ is the hidden layer weight of the j^{th} node in the hidden layer corresponding to the i^{th} node in the input layer; xᵢ is an input value of the i^{th} input node in the input layer; η is a preset parameter learning coefficient; Hⱼ is the hidden layer output result of the j^{th} node in the hidden layer; uⱼₖ is the updated output layer weight of the k^{th} node in the output layer corresponding to the j^{th} node in the hidden layer; uⱼₖ is the output layer weight of the k^{th} node in the output layer corresponding to the j^{th} node in the hidden layer; θₖ
$${θ}_{j}^{′}$$
is the output layer output result of the k^{th} node in the output layer; Yₖ is the label data corresponding to the k^{th} node in the output layer; m is the quantity of nodes in the output layer; hidden layer bias corresponding to the j^{th} node in the hidden layer; θₖ is the updated output layer bias corresponding to the k^{th} node in the output layer; and θₖ is the output layer bias corresponding to the k^{th} node in the output layer.

8. The machine learning-based fuel gas component analysis method according to claim 4, wherein the constraint layer comprises a component total content constraint, a carbon conservation constraint, a carbon-hydrogen ratio constraint, a fuel gas calorific value constraint, a fuel gas density constraint, a fuel gas component upper and lower limit constraint and an alkane component interval constraint; the initial result comprises a fuel gas methane content, a fuel gas ethane content, a fuel gas propane content, and a fuel gas butane content; and the transmitting the initial result to the constraint layer to obtain the prediction result outputted by the constraint layer comprises:
determining the initial result as the prediction result when the initial result satisfies the component total content constraint, the carbon conservation constraint, the carbon-hydrogen ratio constraint, the fuel gas calorific value constraint, the fuel gas density constraint, the fuel gas component upper and lower limit constraint and the alkane component interval constraint at the same time;
otherwise, determining a fuel gas component solution space according to the component total content constraint, the carbon conservation constraint, the carbon-hydrogen ratio constraint, the fuel gas calorific value constraint, the fuel gas density constraint, the fuel gas component upper and lower limit constraint and the alkane component interval constraint; and
performing optimization in the fuel gas component solution space according to the initial result, and determining a solution in the fuel gas component solution space that is closest to the initial result as the prediction result;
wherein the component total content constraint satisfies:
$${C}_{{CH}_{4}} + {C}_{{C}_{2} {H}_{6}} + {C}_{{C}_{3} {H}_{8}} + {C}_{{C}_{4} {H}_{10}} < 100 ;$$
the carbon conservation constraint satisfies:
$${C}_{{CH}_{4}} + 2 {C}_{{C}_{2} {H}_{6}} + 3 {C}_{{C}_{3} {H}_{8}} + 4 {C}_{{C}_{4} {H}_{10}} < ( {C}_{{CO}_{2}} + {C}_{CO} ) \times {V}_{fg} ;$$
the carbon-hydrogen ratio constraint satisfies:
$${A}_{1} \leq \frac{4 {C}_{{CH}_{4}} + 6 {C}_{{C}_{2} {H}_{6}} + 8 {C}_{{C}_{3} {H}_{8}} + 10 {C}_{{C}_{4} {H}_{10}}}{{C}_{{CH}_{4}} + 2 {C}_{{C}_{2} {H}_{6}} + 3 {C}_{{C}_{3} {H}_{8}} + 4 {C}_{{C}_{4} {H}_{10}}} \leq {A}_{2} ;$$
the fuel gas calorific value constraint satisfies:
$${B}_{1} \leq 35.9 {C}_{{CH}_{4}} + 64.4 {C}_{{C}_{2} {H}_{6}} + 93.24 {C}_{{C}_{3} {H}_{8}} + 123.6 {C}_{{C}_{4} {H}_{10}} \leq {B}_{2} ;$$
the fuel gas density constraint satisfies:
$${C}_{1} \leq 0.72 {C}_{{CH}_{4}} + 1.36 {C}_{{C}_{2} {H}_{6}} + 2.01 {C}_{{C}_{3} {H}_{8}} + 2.7 {C}_{{C}_{4} {H}_{10}} \leq {C}_{2} ;$$
the fuel gas component upper and lower limit constraint satisfies:
$${ρ}_{m i n} \leq {C}_{{CH}_{4}} \leq {ρ}_{m ax} ; \\ {σ}_{m i n} \leq {C}_{{C}_{2} {H}_{6}} \leq {σ}_{m ax} ; \\ {τ}_{m i n} \leq {C}_{{C}_{3} {H}_{8}} \leq {τ}_{m ax} ; \\ {φ}_{m i n} \leq {C}_{{C}_{4} {H}_{10}} \leq {φ}_{m ax} ;$$
the alkane component interval constraint satisfies:
$${C}_{{C}_{4} {H}_{10}} \leq {C}_{{C}_{2} {H}_{6}} ; \\ {C}_{{C}_{3} {H}_{8}} \leq {C}_{{C}_{2} {H}_{6}} ;$$
the prediction result satisfies:
$$min f ( {C}_{i} ) = {\sum }_{i = 1}^{4} {( {C}_{i} - {C}_{i}^{′} )}^{2} , {C}_{i} ∈ {R}^{4} ;$$
wherein, C_{CH}_{₄} is the fuel gas methane content; C_{C}_{₂}_{H}_{₆} is the fuel gas ethane content; C_{C}_{₂}_{H}_{₈} is the fuel gas propane content; C_{C}_{₄}_{H}_{₁₀} is the fuel gas butane content; V_{fg} is the acquired volume of a flue gas generated by fuel gas combustion; A₁ is a preset carbon-hydrogen ratio lower limit; A₂ is a preset carbon-hydrogen ratio upper limit; B₁ is a preset fuel gas calorific value lower limit; B₂ is a preset fuel gas calorific value upper limit; C₁ is a preset fuel gas density lower limit; C₂ is a preset fuel gas density upper limit; min ƒ(Cᵢ) is a minimum value of an objective function ƒ(Cᵢ); Cᵢ is a content of an i^{th} component in a fuel gas component prediction result;
$${C}_{i}^{′}$$
is a content of an i^{th} component in a fuel gas component initial result; R⁴ is a four-dimensional fuel gas component solution space; ρₘᵢₙ is a lower limit of the fuel gas methane content; ρₘₐₓ is an upper limit of the fuel gas methane content; σₘᵢₙ is a lower limit of the fuel gas ethane content; σₘₐₓ is an upper limit of the fuel gas ethane content; τₘᵢₙ is a lower limit of the fuel gas propane content; τₘₐₓ is an upper limit of the fuel gas propane content; φₘᵢₙ is a lower limit of the fuel gas butane content; and φₘₐₓ is an upper limit of the fuel gas butane content.

9. A machine learning-based fuel gas component analysis system, comprising:
an acquisition module, configured to acquire the flue gas oxygen content, the flue gas carbon dioxide content, the flue gas carbon monoxide content and the flue gas water content in a flue gas, as well as the fuel gas density, the combustion chamber temperature and the air absolute humidity;
a first processing module, configured to obtain the content of water produced by fuel gas combustion in the flue gas through the water content relationship according to the flue gas carbon monoxide content, the flue gas water content and the air absolute humidity;
a second processing module, configured to obtain the excess air coefficient through the excess air relationship according to the flue gas oxygen content, the flue gas carbon monoxide content and the flue gas carbon dioxide content; and
a prediction module, configured to input the excess air coefficient, the flue gas oxygen content, the flue gas carbon dioxide content, the flue gas carbon monoxide content, the content of water produced by fuel gas combustion in the flue gas, the fuel gas density and the combustion chamber temperature into a trained fuel gas component prediction model, and output a fuel gas component analysis result.

10. An electronic device, comprising a memory and a processor, wherein the memory is configured to have stored a computer program therein; and
the processor is configured to implement the machine learning-based fuel gas component analysis method according to claim 1 while executing the computer program.

11. The electronic device of claim 10, wherein the water content relationship satisfies:
$${C}_{{H}_{2} O}^{′} = {C}_{{H}_{2} 0} - {E}_{{H}_{2} O} \frac{200 - {C}_{CO} - {C}_{{H}_{2} 0}}{200 - {E}_{{H}_{2} O}} ;$$
wherein,
$${C}_{{H}_{2} O}^{′}$$
is the content of water produced by fuel gas combustion in the flue gas;
C_{H}_{₂}₀ is the flue gas water content; E_{H}_{₂}_{O} is the air absolute humidity; and C_{CO} is the flue gas carbon monoxide content.

12. The electronic device of claim 10, wherein the excess air relationship satisfies:
$$α = \frac{β}{β - ( 1 - β ) \frac{{C}_{{O}_{2}} - 0.5 {C}_{CO}}{100 - ( {C}_{{CO}_{2}} + {C}_{{O}_{2}} + {C}_{CO} )}} ;$$
wherein, α is the excess air coefficient; C_{O}_{₂} is the flue gas oxygen content; C_{CO} is the flue gas carbon monoxide content; C_{CO}_{₂} is the flue gas carbon dioxide content; and β is an oxygen content coefficient.

13. The electronic device of claim 10, wherein a training process of the fuel gas component prediction model comprises:
acquiring fuel gas component training data and an initial neural network, the fuel gas component training data comprising input data and label data, and the initial neural network comprising an input layer, a hidden layer, an output layer and a constraint layer;
transmitting the input data to the hidden layer through the input layer to obtain a hidden layer output result;
transmitting the hidden layer output result to the output layer to obtain an initial result outputted by the output layer;
transmitting the initial result to the constraint layer to obtain a prediction result outputted by the constraint layer; and
updating a hidden layer weight, a hidden layer bias, an output layer weight and an output layer bias in the initial neural network according to the prediction result and a comparison result of the corresponding label data to obtain the trained fuel gas component prediction model.

14. The electronic device of claim 13, wherein the hidden layer output result satisfies:
$${H}_{j} = g ( {\sum }_{i = 1}^{n} {w}_{ij} {x}_{i} + {θ}_{j} ) ;$$
wherein, Hⱼ is the hidden layer output result of a j^{th} node in the hidden layer; wᵢⱼ is the hidden layer weight of the j^{th} node in the hidden layer corresponding to an i^{th} node in the input layer; xᵢ is an input value of the i^{th} node in the input layer; θⱼ is a bias corresponding to the j^{th} node in the hidden layer; g( ) is an activation function; and n is the quantity of nodes in the input layer.

15. The electronic device of claim 13, wherein the output layer output result satisfies:
$${O}_{k} = {\sum }_{j = 1}^{l} {u}_{jk} {H}_{j} + {θ}_{k} ;$$
wherein, θₖ is the output layer output result of a k^{th} node in the output layer; uⱼₖ is the output layer weight of the k^{th} node in the output layer corresponding to the j^{th} node in the hidden layer; Hⱼ is the hidden layer output result of the j^{th} node in the hidden layer; θₖ is a bias corresponding to the k^{th} node in the output layer; and 1 is the quantity of nodes in the hidden layer.

16. The electronic device of claim 14, wherein the updated hidden layer weight satisfies:
$${w}_{ij}^{′} = {w}_{ij} + η {H}_{j} ( 1 - {H}_{j} ) {x}_{i} {\sum }_{k = 1}^{m} {u}_{jk} ( {Y}_{k} - {O}_{k} ) ;$$
the updated hidden layer bias satisfies:
$${θ}_{j}^{′} = {θ}_{j} + η {H}_{j} ( 1 - {H}_{j} ) {\sum }_{k = 1}^{m} {u}_{jk} ( {Y}_{k} - {O}_{k} ) ;$$
the updated output layer weight satisfies:
$${u}_{jk}^{′} = {u}_{jk} + η {H}_{j} ( {Y}_{k} - {O}_{k} ) ;$$
the updated output layer bias satisfies:
$${θ}_{k}^{′} = {θ}_{k} + η ( {Y}_{k} - {O}_{k} ) ;$$
wherein,
$${w}_{ij}^{′}$$
is the updated hidden layer weight of the j^{th} node in the hidden layer corresponding to the i^{th} node in the input layer; wᵢⱼ is the hidden layer weight of the j^{th} node in the hidden layer corresponding to the i^{th} node in the input layer; xᵢ is an input value of the i^{th} input node in the input layer; η is a preset parameter learning coefficient; Hⱼ is the hidden layer output result of the j^{th} node in the hidden layer;
$${u}_{j k}^{′}$$
is the updated output layer weight of the k^{th} node in the output layer corresponding to the j^{th} node in the hidden layer; uⱼₖ is the output layer weight of the k^{th} node in the output layer corresponding to the j^{th} node in the hidden layer; θₖ is the output layer output result of the k^{th} node in the output layer; Yₖ is the label data corresponding to the k^{th} node in the output layer; m is the quantity of nodes in the output layer;
$${θ}_{j}^{′}$$
is the updated hidden layer bias corresponding to the j^{th} node in the hidden layer; θⱼ is the layer bias corresponding to the k^{th} node in the output layer; and
$${θ}_{k}^{′}$$
is the output layer bias corresponding to the k^{th} node in the output layer.

17. The electronic device of claim 13, wherein the constraint layer comprises a component total content constraint, a carbon conservation constraint, a carbon-hydrogen ratio constraint, a fuel gas calorific value constraint, a fuel gas density constraint, a fuel gas component upper and lower limit constraint and an alkane component interval constraint; the initial result comprises a fuel gas methane content, a fuel gas ethane content, a fuel gas propane content, and a fuel gas butane content; and the transmitting the initial result to the constraint layer to obtain the prediction result outputted by the constraint layer comprises:
determining the initial result as the prediction result when the initial result satisfies the component total content constraint, the carbon conservation constraint, the carbon-hydrogen ratio constraint, the fuel gas calorific value constraint, the fuel gas density constraint, the fuel gas component upper and lower limit constraint and the alkane component interval constraint at the same time;
otherwise, determining a fuel gas component solution space according to the component total content constraint, the carbon conservation constraint, the carbon-hydrogen ratio constraint, the fuel gas calorific value constraint, the fuel gas density constraint, the fuel gas component upper and lower limit constraint and the alkane component interval constraint; and
performing optimization in the fuel gas component solution space according to the initial result, and determining a solution in the fuel gas component solution space that is closest to the initial result as the prediction result;
wherein the component total content constraint satisfies:
$${C}_{CH 4} + {C}_{{C}_{2} {H}_{6}} + {C}_{{C}_{3} {H}_{8}} + {C}_{{C}_{4} {H}_{10}} < 100 ;$$
the carbon conservation constraint satisfies:
$${C}_{{CH}_{4}} + 2 {C}_{{C}_{2} {H}_{6}} + 3 {C}_{{C}_{3} {H}_{8}} + 4 {C}_{{C}_{4} {H}_{10}} < ( {C}_{{CO}_{2}} + {C}_{CO} ) \times {V}_{fg} ;$$
the carbon-hydrogen ratio constraint satisfies:
$${A}_{1} \leq \frac{4 {C}_{{CH}_{4}} + 6 {C}_{{C}_{2} {H}_{6}} + 8 {C}_{{C}_{3} {H}_{8}} + 10 {C}_{{C}_{4} {H}_{10}}}{{C}_{{CH}_{4}} + 2 {C}_{{C}_{2} {H}_{6}} + 3 {C}_{{C}_{3} {H}_{8}} + 4 {C}_{{C}_{4} {H}_{10}}} \leq {A}_{2} ;$$
the fuel gas calorific value constraint satisfies:
$${B}_{1} \leq 35.9 {C}_{{CH}_{4}} + 64.4 {C}_{{C}_{2} {H}_{6}} + 93.24 {C}_{{C}_{3} {H}_{8}} + 123.6 {C}_{{C}_{4} {H}_{10}} \leq {B}_{2} ;$$
the fuel gas density constraint satisfies:
$${C}_{1} \leq 0.72 {C}_{{CH}_{4}} + 1.36 {C}_{{C}_{2} {H}_{6}} + 2.01 {C}_{{C}_{3} {H}_{8}} + 2.7 {C}_{{C}_{4} {H}_{10}} \leq {C}_{2} ;$$
the fuel gas component upper and lower limit constraint satisfies:
$${ρ}_{min} \leq {C}_{{CH}_{4}} \leq {ρ}_{max} ;$$
$${σ}_{min} \leq {C}_{{C}_{2} {H}_{6}} \leq {σ}_{max} ;$$
$${τ}_{min} \leq {C}_{{C}_{3} {H}_{8}} \leq {τ}_{max} ;$$
$${φ}_{min} \leq {C}_{{C}_{4} {H}_{10}} \leq {φ}_{max} ;$$
the alkane component interval constraint satisfies:
$${C}_{{C}_{4} {H}_{10}} \leq {C}_{{C}_{2} {H}_{6}} ;$$
$${C}_{{C}_{3} {H}_{8}} \leq {C}_{{C}_{2} {H}_{6}} ;$$
the prediction result satisfies:
$$min f ( {C}_{i} ) = {\sum }_{i = 1}^{4} {( {C}_{i} - {C}_{i}^{′} )}^{2} , {C}_{i} ∈ {R}^{4} ;$$
wherein, C_{CH}_{₄} is the fuel gas methane content; C_{C}_{₂}_{H}_{₆} is the fuel gas ethane content; C_{C}_{₃}_{H}_{₈} is the fuel gas propane content; C_{C}_{₄}_{H}_{₂}, is the fuel gas butane content; V_{fg} is the acquired volume of a flue gas generated by fuel gas combustion; A₁ is a preset carbon-hydrogen ratio lower limit; A₂ is a preset carbon-hydrogen ratio upper limit; B₁ is a preset fuel gas calorific value lower limit; B₂ is a preset fuel gas calorific value upper limit; C₁ is a preset fuel gas density lower limit; C₂ is a preset fuel gas density upper limit; min ƒ(Cᵢ) is a minimum value of an objective function ƒ(Cᵢ); Cᵢ is a content of an i^{th} component in a fuel gas component prediction result;
$${C}_{i}^{′}$$
is a content of an i^{th} component in a fuel gas component initial result; R⁴ is a four-dimensional fuel gas component solution space; ρₘᵢₙ is a lower limit of the fuel gas methane content; ρₘₐₓ is an upper limit of the fuel gas methane content; σₘᵢₙ is a lower limit of the fuel gas ethane content; σₘₐₓ is an upper limit of the fuel gas ethane content; τₘᵢₙ is a lower limit of the fuel gas propane content; τₘₐₓ is an upper limit of the fuel gas propane content; φₘᵢₙ is a lower limit of the fuel gas butane content; and φₘₐₓ is an upper limit of the fuel gas butane content.
